(19) United States
(12) Patent Application Publication
(10) Pub. No.: US 2023/0285761 A1
(43) Pub. Date: Sep. 14, 2023

(54) METHOD AND DEVICE FOR MONITORING CAPACITOR CHARGING AS A METRIC FOR LONG-TERM CAPACITOR HEALTH IN AN IMPLANTABLE DEVICE

(71) Applicant: Pacesetter, Inc., Sylmar, CA (US)

(72) Inventors: Jeffery Crook, Belmont, CA (US); R. Jason Hemphill, Sunset, CA (US); David Bowen, Taylors, SC (US)

(21) Appl. No.: 18/163,442

(22) Filed: Feb. 2, 2023

Related U.S. Application Data
(60) Provisional application No. 63/317,443, filed on Mar. 7, 2022.

(51) Int. Cl. A61N 1/378; A61N 1/39

(52) U.S. Cl. CPC A61N 1/3782; A61N 1/3956

(57) ABSTRACT

System, methods and devices are provided for identifying a failure state of a charge storage device. The system includes a charge storage device within an implantable medical device. An energy supply is configured to charge the charge storage device during a charging operation. A monitoring circuit is coupled to the charge storage device and is configured to collect voltage level measurements across the charge storage device at multiple points in time during the charging operation. Responsive to execution of program instructions, a processor collects the voltage level measurements across the charge storage device. The charge storage device exhibits a charge profile in which a voltage level across the charge storage device changes over the charging operation. The processor analyzes the voltage level measurements to identify a failure signature in the charge profile and generates an output indicative of a failure state for the charge storage device based on the analysis.

### CROSS-REFERENCE TO RELATED APPLICATIONS

This application claims priority to U.S. Provisional Pat. Application No. 63/317,443, filed 07-March-2022, titled METHOD AND DEVICE FOR MONITORING CAPACITOR CHARGING AS A METRIC FOR LONG-TERM CAPACITOR HEALTH IN AN IMPLANTABLE DEVICE”. The subject matter of the provisional application is expressly incorporated herein by reference in its entirety.

### BACKGROUND

Embodiments of the present disclosure generally relate to methods and devices to detect a failure state of a charge storage device within a medical device.

A variety of implantable medical devices (IMDs) and portable external electronic medical devices (EMDs) are utilized today in connection with a wide array of health-related topics. Some implantable and external portable medical devices utilize a charge storage device that may include one or more high voltage capacitors that are utilized to delivery therapy. An IMD generally includes a battery, as the energy source, to provide power over the life of the IMD. The battery supplies power for various electronic circuits, for telemetry circuits, for delivery of low voltage pacing therapies and for mid/high voltage shock therapies.

Heretofore, processes have been proposed to monitor battery usage and to track/predict a remaining life of the battery or to predict an early battery depletion condition. For example, a method has been proposed to determine a charge consumption drawn externally from a battery cell by the device for a select period of time. The method obtains a measured cell voltage for the battery cell and calculates a projected cell voltage based on the charge consumption and usage conditions. The method declares an early battery depletion condition based on a relation between the measured and projected cell voltages.

However, the foregoing and other solutions only account for certain aspects of a condition/life of an IMD. The foregoing and other solutions have not addressed a health/condition of the charge storage device(s) within the IMD or portable EMD. The charge storage device (CSD) (e.g., one or more capacitors) may exhibit partial failures over the course of a life of the CSD. Depending upon a nature of the failure, the failure could cause inferior performance for the CSD, shorten a life of the CSD and/or cause undue power draw upon the battery that in turn shortens an overall life of the IMD/EMD.

In certain instances, charge storage devices have exhibited premature failure for various reasons. Although premature charge storage device failure is an uncommon event in implantable medical devices, it does occur. Charge storage device failure, such as within an implantable defibrillator and/or subcutaneous implantable cardioverter defibrillator, may result in lack of adequate shocking voltage when needed by the patient, and/or cause the charge storage device to take an excessive amount of time to charge.

By way of example, one mechanism of charge storage device failure can be caused by chemical contaminants within the capacitor(s) themselves, primarily elements in the Halide group, or to a lesser degree, Iron or Copper if taken up by the anode dielectric oxide, in the case of an Aluminum electrolytic capacitor. Other possible mechanisms are anode/cathode cracks/detachment, separator failure. Often, the root-cause of the malfunction manifests as chronic scintillation events, the requisite energy being derived from a partial discharge of the capacitor(s) stored energy. Periodic reforming, by charging the capacitor(s) to a high voltage will often repair any damage caused by a small anode crack, or at least temporarily, a scintillation event, in addition to minor benign endemic degradation to the dielectric due chiefly to the necessary small water content in the electrolyte. However, the capacitor(s) can sustain some level of damage due to the failure that can impact the longevity and performance.

A need remains for methods and devices that monitor deterioration or other latent issues that can shorten the lifespan and/or limit the capabilities of the charge storage device.

### SUMMARY

In accordance with embodiments herein a system comprises a charge storage device within an implantable medical device (IMD). An energy supply is configured to charge the charge storage device during a charging operation. A monitoring circuit is coupled to the charge storage device and is configured to collect voltage level measurements across the charge storage device at multiple points in time during the charging operation. Responsive to execution of program instructions, a processor collects the voltage level measurements across the charge storage device. The charge storage device exhibits a charge profile in which a voltage level across the charge storage device changes over the charging operation. The processor analyzes the voltage level measurements to identify a failure signature in the charge profile and, in response to identifying the failure signature, generates an output indicative of a failure state for the charge storage device based on the analysis.

Optionally, while actively charging the charge storage device, the failure signature may represent a partial discharge in the charge profile, and the partial discharge may be expressed by a decrease in the voltage level measurements followed by a recovery in the voltage level measurements.

Optionally, when the charge storage device is in a non-failure state, the charge profile may exhibit a voltage level that monotonically increases throughout a period of time in which the charge storage device is actively being charged, and when the charge storage device is in a failure state, the charge profile may exhibit one or more voltage drops during the period of time in which the charge storage device is actively being charged.

Optionally, to analyze the voltage level measurements, the processor may be configured to calculate a rate of change per unit of time in the charge profile and to determine when the rate of change has a negative slope. The processor may be configured to calculate voltage changes between successive first and second voltage level measurements and to determine when the second voltage level measurement is below the first voltage level measurement.

Optionally, the charge profile may include an active segment and an inactive segment. The active segment may correspond to a period of time during the charging operation in which the charge storage device is actively being charged, and the inactive segment may correspond to a period of time during the charging operation in which the charge storage device is not being charged.

Optionally, the IMD may house the energy supply and the monitoring circuit. The system may further comprise an external device that may be configured to communicate with the IMD, and at least one of the IMD or the external device may include a memory and the processor. The output may include a communication from the IMD that indicates that one or more capacitors of the charge storage device is experiencing the failure state. The charge storage device may include one or more capacitors in the IMD, and the failure state for the one or more capacitors may include at least one of i) a loss of a dielectric layer of a capacitor, ii) corrosion detected, iii) oxide formation, iv) at least one of iron, chlorine, bromine, iodine, or copper contamination, or v) short circuit between adjacent dielectric layers.

In accordance with embodiments herein, a method, under control of one or more processors, is configured with specific executable instructions to use an energy supply to charge a charge storage device, within an IMD, during a charging operation. The method directs a monitoring circuit to collect voltage level measurements across the charge storage device at multiple points in time during the charging operation. The charge storage device exhibits a charge profile in which a voltage level across the charge storage device changes over the charging operation. The method analyzes the voltage level measurements to identify a failure signature in the charge profile and, in response to identifying the failure signature, generates an output indicative of a failure state for the charge storage device based on the analysis.

Optionally, the failure signature may represent one of an increase in time or a decrease in time for the charge profile to reach a predetermined voltage level compared to a baseline charge time to reach the predetermined voltage level.

Optionally, the method may adjust the time for the charge profile to reach the predetermined voltage level based on at least one of voltage and equivalent series resistance associated with the energy supply and may compare the adjusted time to the baseline charge time.

Optionally, while the method actively charges the charge storage device, the failure signature may represent a partial discharge in the charge profile. The partial discharge may be expressed by a decrease in the voltage level measurements, followed by a recovery in the voltage level measurements.

Optionally, when the charge storage device is in a non-failure state, the charge profile may exhibit a voltage level that monotonically increases throughout a period of time in which the charge storage device is actively being charged, and when the charge storage device is in a failure state, the charge profile may exhibit one or more voltage drops during the period of time in which the charge storage device is actively being charged.

Optionally, the analyzing operation may calculate a rate of change per unit of time in the charge profile and determine when the rate of change has a negative slope. The analyzing operation may calculate voltage changes between successive first and second voltage level measurements and determine when the second voltage level measurement is below the first voltage level measurement.

Optionally, the charge profile may include an active segment and an inactive segment. The active segment may correspond to a period of time during the charging operation in which the charge storage device is actively being charged and the inactive segment may correspond to a period of time during the charging operation in which the charge storage device is not being charged. The method may direct the monitoring circuit to collect second voltage level measurements across the charge storage device at multiple points during the inactive segment and may analyze the second voltage level measurements to identify a second failure signature in the charge profile. The second failure signature may represent an increased rate of change compared to a baseline rate of change.

Optionally, the method may direct the monitoring circuit to collect third voltage level measurements across the charge storage device at multiple points during a second inactive segment that is subsequent to the inactive segment and may average the second and third voltage level measurements across the inactive segment and the second inactive segment.

### BRIEF DESCRIPTION OF THE DRAWINGS

FIG. 1 illustrates a simplified block diagram of a system for charging and determining the health of a charge storage device in accordance with embodiments herein.

FIG. 2 illustrates a method for determining a baseline performance of the charge storage device in accordance with embodiments herein.

FIG. 3 shows several charge profiles indicating charge time performance of different conditions of the charge storage device in accordance with embodiments herein.

FIG. 4 illustrates a method for determining charge time performance of the charge storage device while the associated implantable medical device (IMD) is implanted in a patient in accordance with embodiments herein.

FIG. 5 illustrates a method for measuring a continuity of capacitor voltage of the charge storage device during a charging task in accordance with embodiments herein.

FIG. 6 illustrates a charge profile that has multiple failure states associated with voltage drops (e.g., scintillation voltage drops) in accordance with embodiments herein.

FIG. 7 illustrates a method for measuring health of the charge storage device by measuring leakage (e.g., voltage drop, leakage current, etc.) during a charging task in accordance with embodiments herein.

FIG. 8 illustrates a charge profile wherein the charge storage device is charged several successive times with decay periods therebetween during which the charge storage device is not being charged in accordance with embodiments herein.

FIG. 9 illustrates a method for measuring health of the charge storage device by measuring micro-decay during a pulsed charging task at high voltage in accordance with embodiments herein.

FIG. 10 illustrates a charge profile wherein a series of quick pulses at peak voltage are applied to the charge storage device in accordance with embodiments herein.

FIG. 11 shows an exemplary IMD that is implanted into the patient as part of an implantable cardiac system in accordance with embodiments herein.

FIG. 12 illustrates a functional block diagram of an external device that is operated in accordance with embodiments herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

### Terms

The term “task” refers to predefined operations to be performed by a medical device over a useful life of the medical device. The predefined operations may be in connection with delivering therapy, monitoring signals, communication with other devices, self-diagnostics, internal maintenance, and the like. Nonlimiting examples of device related tasks include reforming capacitors (e.g., capacitor maintenance), delivering shocks or other therapy, performing telemetry operations, transmitting and receiving RF communications, collecting voltage level measurements over segments of time during which the capacitor(s) in a charge storage device are being charged (e.g., active segment) and during which the capacitor(s) are not being charged (e.g., inactive segment).

The term “active segment” shall mean a state and/or duration of time during which the capacitor(s) in a charge storage device are being charged, while the term “inactive segment” shall mean a state and/or duration of time during which the capacitor(s) are not being charged.

The term “failure state”, when describing a charge storage device, shall mean a state in which the charge storage device still operates but may not be operating in a normal or healthy manner and/or may not operate normally for the expected life or duration of the device. For example, the failure state can indicate a potential to fail or a partial failure, and can be used together with other data, such as other logged failure states over time, to determine that the health of the charge storage device is beginning to erode and/or may be at a level that service or replacement of the medical device may be considered.

The term “health” and “healthy”, when describing a charge storage device, shall mean a general level of capability and functionality of the charge storage device. For example, a “healthy” charge storage device may not exhibit one or more of the failure states described herein. In some cases, the health storage device can exhibit one or more of the failure states, but still remain in a healthy state. However, a charge storage device can begin to experience an erosion of health (e.g., indications that the associated device may not experience a full life span and/or require service) due to various breakdowns and/or malfunctions, such as but not limited to: an increase or decrease in charge time performance, voltage drops that occur during a charging task, increased voltage loss during a time period when the charge storage device is not being actively charged, excess decay calculated over a series of micro-pulses at high voltage, collected data fails a comparison to one or more threshold(s), and the like.

The term “failure”, when used to describe a state or condition of a charge storage device, shall mean that the charge storage device has experienced one or more of various breakdowns or malfunction events described herein and otherwise known in the field. It is understood that a charge storage device that is in a failure state still functions, albeit at a lesser performance level.

The term “failure signature”, when used to describe a state of condition of a charge storage device, shall mean that an unexpected result has been detected which may indicate one or more of various breakdowns or malfunction events. Failure signatures as used herein include but are not limited to: the charge storage device reaching a predetermined voltage level earlier or later than expected, a decrease in voltage and/or slope of the voltage curve when an increase in voltage is expected, a rate of change per unit of time that is greater than or lesser than expected, collected voltage level measurements and/or current measurements that exceed a threshold when compared to a baseline and/or previous measurements, and/or averaged collected measurements that exceed a threshold when compared to a baseline and/or previous measurements.

The terms “normal state” and “non-failure state”, when describing a charge storage device, shall mean a state in which the charge storage device is not experiencing a failure.

The term “charging operation” shall mean that an energy supply is electrically coupled to a charge storage device, such as with a charge circuit, and is transferring energy to the charge storage device, (e.g., “actively charging” or “actively charge” the charge storage device).

The term “reforming” shall mean the process of applying a high voltage level across the capacitor(s) of the charge storage device to repair faults in chemistry of the capacitor(s) (e.g., re-growing oxide in grooves of aluminum foil). By way of example, embodiments herein may implement all or portions of the processes and structures for capacitor reforming as described in U.S. Pat. 7,848,804, titled “Apparatus and related Methods for Capacitor Reforming”, issuing Dec. 7, 2010, U.S. Pat. 9,976,226, titled “Method of Stressing Oxides”, issuing May 22, 2018, U.S. Pat. 7,570,995, titled “Method for Reforming a Capacitor in an Implantable Medical Device”, issuing Aug. 4, 2009, and U.S. Application 2010/0114235, titled “Hybrid Battery System for Implantable Cardiac Therapy Device”, publishing May 6, 2010, the complete subject matter of which are incorporated herein by reference in their entireties.

The terms “cardiac activity signal”, “cardiac activity signals”, “CA signal” and “CA signals” (collectively “CA signals”) are used interchangeably throughout to refer to measured signals indicative of cardiac activity by a region or chamber of interest. For example, the CA signals may be indicative of impedance, electrical or mechanical activity by one or more chambers (e.g., left or right ventricle, left or right atrium) of the heart and/or by a local region within the heart (e.g., impedance, electrical or mechanical activity at the AV node, along the septal wall, within the left or right bundle branch, within the purkinje fibers). The cardiac activity may be normal/healthy or abnormal/arrhythmic. An example of CA signals includes EGM signals. Electrical based CA signals refer to an analog or digital electrical signal recorded by two or more electrodes, where the electrical signals are indicative of cardiac activity. Heart sound (HS) based CA signals refer to signals output by a heart sound sensor such as an accelerometer, where the HS based CA signals are indicative of one or more of the S1, S2, S3 and/or S4 heart sounds. Impedance based CA signals refer to impedance measurements recorded along an impedance vector between two or more electrodes, where the impedance measurements are indicative of cardiac activity.

The terms “high-voltage shock” and “HV shock” refer to defibrillation stimulus delivered at an energy level sufficient to terminate a defibrillation episode in a heart, wherein the energy level is defined in Joules (J) to be 40J or more and/or the energy level is defined in terms of voltage (V) to be 750V or more.

The terms “medium-voltage shock” and “MV shock” refer to defibrillation stimulus delivered at an energy level sufficient to terminate a defibrillation episode in a heart, wherein the energy level is defined in Joules, pulse width, and/or maximum charge voltage. An MV shock from an implantable medical device (IMD) with a transvenous lead will have a different maximum energy and/or charge voltage than an MV shock from a subcutaneous IMD with a subcutaneous lead. In connection with an IMD having a transvenous lead, the terms medium voltage shock and MV shock refer to defibrillation stimulation that has an energy level that is no more than 25 J, and more preferably 15 J - 25 J and/or has a maximum voltage of no more than 500 V, preferably between 100 V - 475 V and more preferably between 400 V - 475 V.

The terms “obtain” and “obtaining”, as used in connection with data, signals, information and the like, include at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The terms “processor,” “a processor”, “one or more processors”, and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration, and the like.

The term “real-time” refers to a time frame contemporaneous with normal or abnormal episode occurrences. For example, a real-time process or operation would occur during or immediately after (e.g., within minutes or seconds after) a cardiac event, a series of cardiac events, an arrhythmia episode, and the like. For example, the term “real-time” may refer to a time period substantially contemporaneous with an event of interest. The term “real-time,” when used in connection with collecting and/or processing data utilizing an IMD, shall refer to processing operations performed substantially contemporaneous with a physiologic event of interest experienced by a patient. By way of example, in accordance with embodiments herein, cardiac activity signals are analyzed in real time (e.g., during a cardiac event or within a few minutes after the cardiac event).

The terms “charge storage device”, “single charge storage capacitor”, “single capacitor”, “capacitors”, “capacitor(s)”, and “capacitor bank” shall mean that a physical component is utilized to perform the corresponding operation or task (e.g., charge to a predetermined voltage level, retain a charge, deliver a shock, maintain a defined initial or later voltage), and shall expressly include the use of one physical component and/or more than one physical component. For example, included configurations can have two or more physical capacitors coupled in parallel or series with one another, or utilized independently to deliver corresponding portions of a shock. Other included configurations can have one physical capacitor.

The term “subcutaneous” shall mean below the skin, but not intravenous. For example, a subcutaneous electrode/lead does not include an electrode/lead located in a chamber of the heart, in a vein on the heart, or in the lateral or posterior branches of the coronary sinus.

### System Overview

In accordance with new and unique aspects herein, methods and devices are described that determine the health of the capacitor(s) that are and/or will be used within an implantable device. The health of the capacitor(s) can be determined i) before the capacitor(s) are installed within the implantable device as a charge storage device, ii) after the capacitor(s) are installed in the implantable device, but before the device is implanted in a patient (e.g., final safety check), iii) after the capacitor(s) have been stored for a period of time, but before the device is implanted in a patient, iv) by a clinician while the device is implanted or partially implanted in a patient, v) while the device is implanted or partially implanted in the patient without intervention or direction from an outside person, device, or system, vi) on a regular schedule, on-demand, and/or in response to a task result, vii) simultaneous with one or more other tasks associated with the capacitor(s), viii) using equipment/components external to the device, and/or ix) using components internal to the device.

The methods and devices can evaluate acquired data and compare the acquired data to expected and/or baseline data (e.g., nominal) and/or previously acquired curves, and/or data sourced from other similar devices. The comparisons can provide the technical benefit of implying or determining the health of the capacitor(s) at multiple points during the lifespan of the charge storage device and/or IMD. Knowing the health of the charge storage device at various points in time can improve the care of patients, facilitating removal, replacement, and/or service of the IMD in a timely, scheduled manner and thus eliminating the need for an emergency procedure.

At least portions of the methods and embodiments disclosed herein can be accomplished simultaneously with each other and/or with the capacitor reforming task. The reforming task can occur on a regular schedule such as every few months (e.g., 3, 6, 9, 12 months) or on demand.

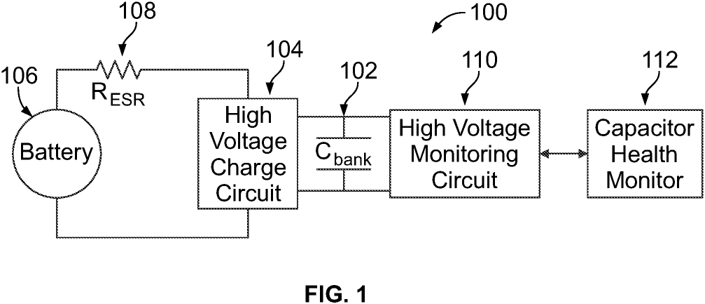

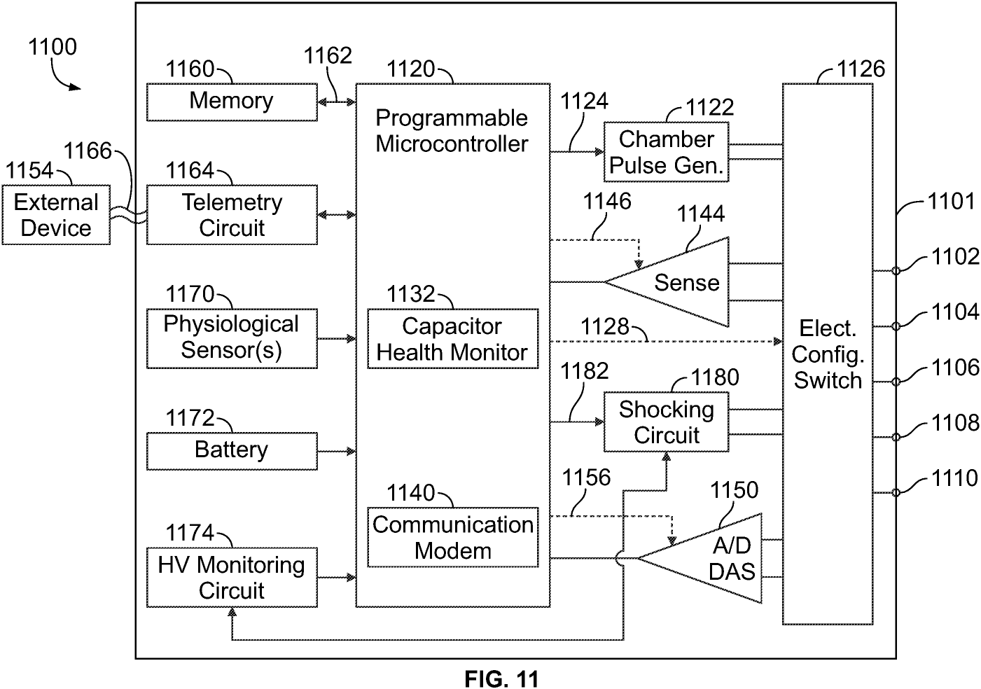

FIG. 1 illustrates a simplified block diagram of a system 100 for determining the health of a charge storage device 102 in accordance with embodiments herein. In some embodiments the system 100 can be within an IMD and/or subcutaneous IMD as discussed further below in FIG. 11. In other embodiments, the system 100 can be wholly or partially outside the IMD, such that the health of the charge storage device 102 can be determined before the charge storage device 102 is included within the IMD.

Referring to FIG. 1, the charge storage device (CSD) 102 can be a single capacitor or a capacitor bank that includes more than one capacitor. The CSD 102 is configured to deliver HV shocks and/or MV shocks in connection with treating an arrhythmia, such as fibrillation and/or a tachycardia. The capacitor(s) can be, for example, aluminum electrolytic capacitors or other type. The CSD 102 is electrically coupled to a high voltage (HV) charge circuit 104, during a charging operation. The HV charge circuit 104 is connected to an energy supply 106 (e.g., battery). The HV charge circuit 104 and/or energy supply 106 may be internal or external with respect to the implantable device. The energy supply 106 is capable of charging the CSD 102 up to at least 900 V. The resistance R_{ESR} 108 indicates equivalent series resistance of the energy supply 106.

A HV monitoring circuit 110 measures a voltage level across the CSD 102 a plurality of times at regular or predetermined time intervals during various operations (e.g., capacitor reforming task, tasks to measure elapsed time to a peak or predetermined voltage, tasks to measure continuity of voltage changes, tasks to measure leakage current, etc.). A capacitor health monitor 112 receives or collects and stores the voltage level measurements. In some embodiments, the capacitor health monitor 112 can supply instructions to the HV monitoring circuit 110 to instruct the circuit 110 to measure the voltage level. The capacitor health monitor 112 uses various algorithms, comparisons, etc., to determine whether the voltage level measurements indicate that the CSD 102 is experiencing a failure state or a non-failure state. For example, the capacitor health monitor 112 can calculate a derivative, double derivative, etc., to determine how the graph of a function is curved. The HV monitoring circuit 110 and capacitor health monitor 112 can be implemented in one or more of hardware, firmware, and software, and can include an analog to digital converter. For example, the capacitor health monitor 112 can be implemented, as a circuit, in hardware or firmware that communicates with one or more processors to provide collected voltage level measurements to the one or more processors for subsequent analysis. Additionally or alternatively, the capacitor health monitor 112 may be implemented as a software module executable by the one or more processors to direct operation of the HV monitoring circuit 110. As discussed above, while a failure state can in some cases indicate a non-functional state of the device, a failure state also includes a degradation of capacity/capability of the device while the device is still fully capable of operation within normal parameters.

If the CSD 102 is experiencing a failure state, an alert may be sent, such as to a clinician, an application (“App”) on a user’s phone associated with the IMD, a manufacturer, service provider, etc. For example, the methods and systems can provide one or more indicators to inform the patient, and/or health care professionals of a failure state. Non-limiting examples of warning indicators include vibration and/or sounds emitted by a medical device. Other non-limiting examples include transmitting warnings wirelessly (e.g., Bluetooth, WiFi, 3G, 4G, 5G) to another electronic device, such as a patient’s personal digital devices (e.g., cell phones, tablet device, laptop computer, etc.), a bedside monitoring device and the like. The receiving device may then convey a visual and/or audible indication to the patient and/or health care professional.

In accordance with embodiments herein, the methods and systems for determining a failure state may be implemented on one or more external devices operating alone or in combination with the battery-powered medical device. For example, the methods described herein may be implemented in hardware and/or software loaded on an external device such as the Merlin@Home™ device or a similar device. The method can compare measured charge storage device performance against parameters saved on the external device, where data space is less limited as compared to memory capacity of implantable medical devices.

The following methods, calculations, and/or measurements can be performed simultaneously, serially, on the same periodic schedule, or on a different schedule as each other or other additional tasks. For example, the methods, calculations, and/or measurements can be performed in connection with reforming the capacitor(s), monitoring signals, communicating with other devices, self-diagnostics, internal maintenance, delivering shocks or other therapy, performing telemetry operations, transmitting and receiving RF communications, and the like.

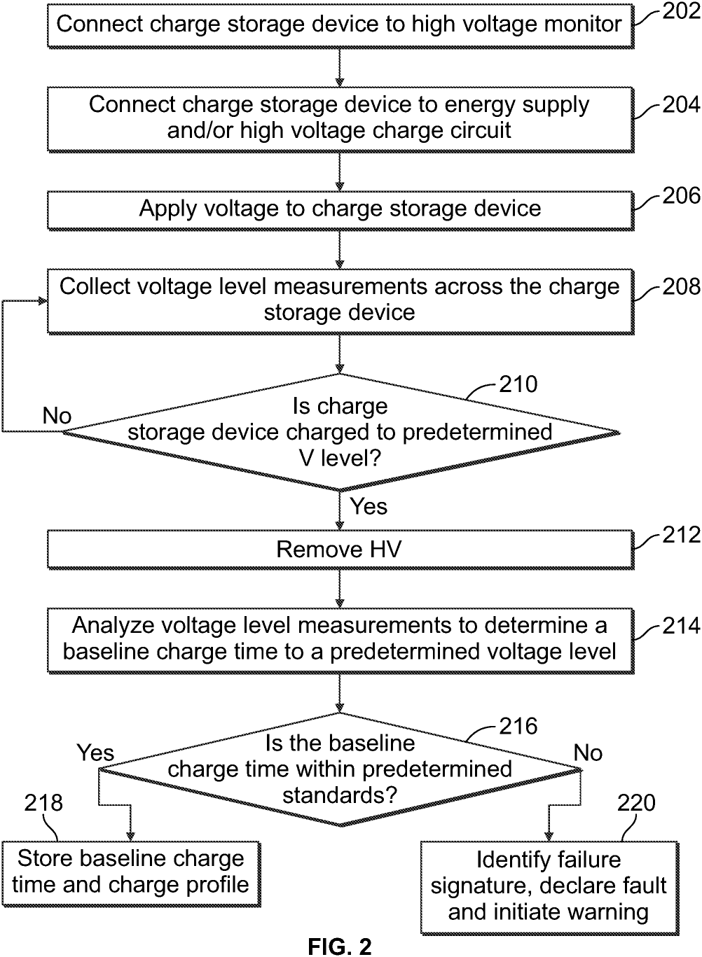

FIG. 2 illustrates a method for determining a baseline performance of the CSD 102 of FIG. 1 in accordance with embodiments herein. The operations of FIG. 2 may be implemented by hardware, firmware, circuitry and/or one or more processors housed partially and/or entirely within an IMD, a local external device, remote server or more generally within a healthcare system. Optionally, the operations of FIG. 2 may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors. In some embodiments, the baseline performance is determined prior to implanting the associated IMD into a patient. For example, in some cases, the CSD 102 can experience decay while in storage. To ensure that the CSD 102 performs to the desired specifications, the CSD 102 can be tested with several testing algorithms as discussed herein.

The baseline performance can include, but is not limited to, an elapsed time to achieve a predetermined voltage level and indicates a “normal” or initial rate of change that does not include a negative slope. The operations in the method of FIG. 2 may be implemented in whole or in part by a portable medical device carried by a clinician or patient, an IMD, an external device, a remote server, a workstation, or a combination thereof.

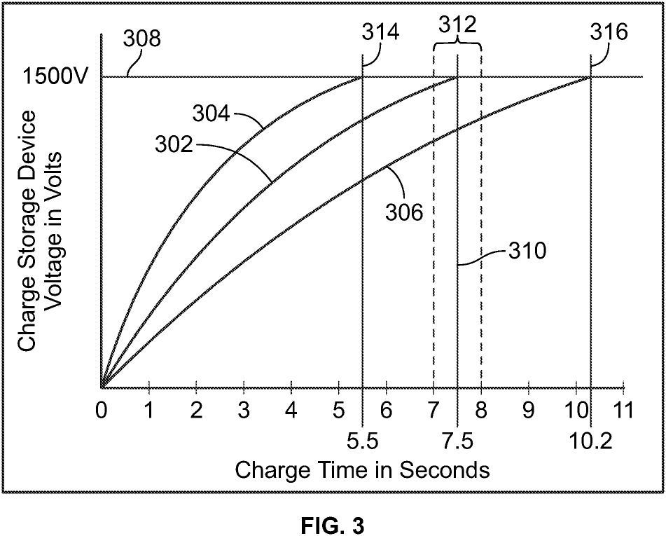

FIG. 3 shows several curves indicating charge profiles of various states and/or conditions of the CSD 102 in accordance with embodiments herein. The horizontal axis plots charge time of the CSD 102 in seconds, while the vertical axis plots the voltage of the CSD 102 in volts. The starting voltage level at time equals 0 seconds can be around 0-20 V but is not so limited (e.g., remnant charge may not be depleted on the HV capacitor). It is recognized that the voltages or shapes of the charge profiles illustrated in the Figures herein are examples and are not limited. FIGS. 2 and 3 will be discussed together.

At 202 of FIG. 2, the CSD 102 is electrically coupled to the HV monitoring circuit 110 and at 204, the CSD 102 is electrically coupled to the energy supply 106 and/or HV charge circuit 104 as required by the charging system configuration. For example, one or more processors (e.g., within the IMD) can direct the connections of 202 and 204 through one or more switches as needed and in any order. In some embodiments, the CSD 102 may already be connected (e.g., within the IMD) to the HV charge circuit 104 and energy supply 106, and thus the system loss through these components may be known through other tests. The energy supply data (e.g., battery voltage, R_{ESR} 108 effect) can also be known with other systems used to conduct the testing procedure.

At 206, one or more processors direct the HV charge circuit 104 to apply voltage to the CSD 102 in a charging operation or charging task. For example, the HV charge circuit 104 is transferring energy, such as from the energy supply 106, to actively charge the CSD 102. In some embodiments, the voltage is applied at a level such as 750 V to 1500 V, to accomplish a reforming task on the capacitor(s) of the CSD 102. For example, the reforming task may be implemented in accordance with one or more of the reforming processes described in the patents and applications referenced herein.

At 208, the HV monitoring circuit 110 collects voltage level measurements across the CSD 102 as the HV charge circuit 104 charges the CSD 102 during a charging operation. The voltage level measurements can be taken at regular intervals (e.g., 1-20 times per second, or about 10 times per second). For example, the HV monitoring circuit 110 may collect the voltage level measurements under direction of the capacitor health monitor 112 (when implemented in hardware or firmware) and/or a capacitor health monitoring module 112 (when implemented in software executable by the one or more processors).

In a healthy CSD 102, the voltage level over time will increase monotonically. Turning to FIG. 3, charge profile 302 is representative of a monotonically increasing voltage level. As used herein, the term “monotonically increasing” means that the slope (DV/DT) of the charge profile 302 is always positive and that there are no slope sections that are decreasing or zero.

At 210 of FIG. 2, the one or more processors determine whether the voltage level measurement meets or exceeds a predetermined voltage level 308, such as, for example, 1500V (indicated with a horizontal line) and/or predetermined energy level (J). If the CSD 102 is charged to the predetermined voltage level 308, at 212, the one or more processors can remove the energy supply 106 from the CSD 102. For example, a voltage level of 900 V can be a level used to apply a safety shock to a patient.

At 214, the one or more processors analyze the voltage level measurements to determine a baseline charge time 310 (indicated with a vertical line) to the predetermined voltage level 308. In the example of FIG. 3, the charge profile 302 reached the predetermined voltage level 308 at 7.5 seconds.

At 216, the one or more processors determine whether the baseline charge time is within a threshold range 312 (indicated by vertical dotted lines) (e.g., predetermined standards). For a healthy CSD 102, the time to reach the predetermined voltage level 308 can be within a threshold range 312 (e.g., 7 seconds to 8 seconds), but the embodiments are not so limited. If the acceptable parameters for a new device ready to be implanted into a patient are within the threshold range 312, the CSD 102 is healthy and the CSD 102 is in a normal or non-failure state. In some embodiments the baseline charge time 310 can also be compared to a previous baseline charge time that was acquired, such as before the device was prepared and placed in storage.

At 218, the one or more processors store the baseline charge time 310. The baseline charge time 310, and in some cases the acquired voltage over time measurements (e.g., charge profile(s)), can be stored locally within the IMD, transferred to an external computer, external system, and the like, and can be used in the future to compare subsequent voltage over time measurements. The baseline charge time 310 is unique to the particular CSD 102 and may also include parameters/losses of an associated IMD. In the example of FIG. 3, the charge profile 302 represents the capacity of the CSD 102 at the beginning of its life (e.g., healthy).

Returning to 216, if the one or more processors determine that the baseline charge time is outside of the threshold range 312, at 220 the one or more processors declare the associated baseline charge time to be a failure signature and can initiate a warning to advise that the CSD 102 has a fault or is in a failure state. To initiate a warning, a communication can be output or transmitted to inform the manufacturer, technician, and/or health care professionals, etc., of the failure state. In some embodiments, if the CSD 102 is identified as faulty, it may be discarded and/or removed from the IMD before the IMD is implanted in the patient.

Referring to FIG. 3, charge profile 304 represents a monotonically increasing curve that reaches the predetermined voltage level 308 earlier than expected (e.g., has increased speed) at charge time 314 (as indicated with a vertical line at approximately 5.5 seconds), which occurs outside of the threshold range 312 (associated with the vertical line at 7.5 seconds (e.g., baseline charge time 310)). This may be the case when a portion of the CSD 102 (e.g., plate or winding of a capacitor) has failed. Therefore, the CSD 102 now stores less energy and over the expected lifetime of the device the CSD 102 may not retain the ability to charge to the predetermined voltage level 308. This can compromise patient safety.

Charge profile 306 represents a monotonically increasing curve that reaches the predetermined voltage level 308 later than expected at charge time 316 (indicated with a vertical line). This may be the case when the CSD 102 has degraded in some manner. If the CSD 102 takes too long to charge, the shocking functionality of the IMD may not be achieved during an acceptable window of time.

In the aforementioned cases of the charge profiles 304 and 306, the health care provider and/or manufacturer can identify charge storage devices 102 that won’t provide or are unlikely to provide the full expected lifespan of the IMD, thus providing the technical benefit of identifying devices that should not be implanted into a patient as the device will require maintenance and/or removal prior to the expected end of life of the device. This rejection applies similarly to any CSD 102 with a charge profile (not shown) that fails to charge to the predetermined voltage level 308.

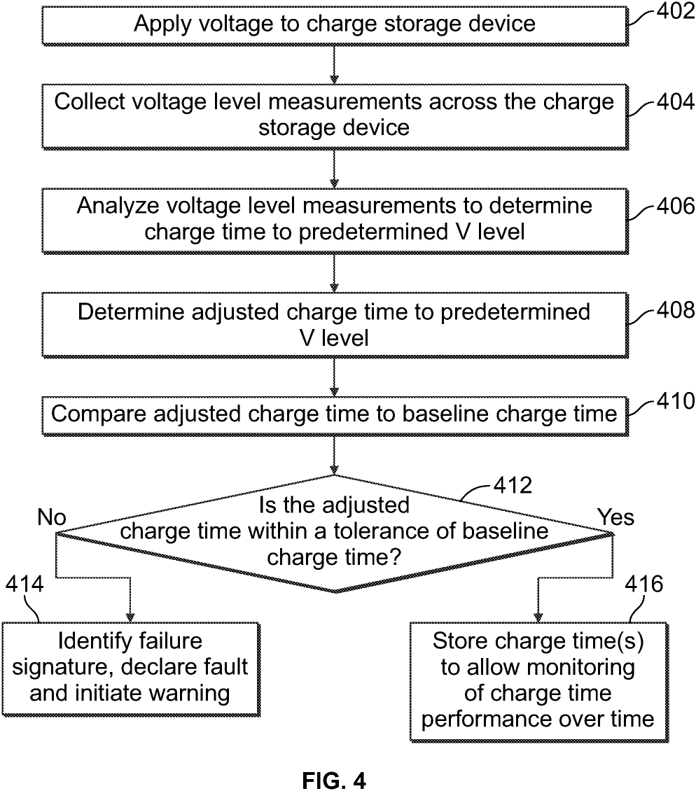

FIG. 4 illustrates a method for determining charge time performance of the CSD 102 of FIG. 1 while the associated IMD is implanted in a patient in accordance with embodiments herein. The operations of FIG. 4 may be implemented by hardware, firmware, circuitry and/or one or more processors housed partially and/or entirely within an IMD, a local external device, remote server or more generally within a healthcare system. Optionally, the operations of FIG. 4 may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors. For example, the charge time performance can be monitored over a period of years. In some embodiments the charge time performance can provide a measurement of health of the CSD 102, and thus of the capacitor(s) within the CSD 102.

At 402, the one or more processors apply voltage to the CSD 102 in a charging operation. For example, the HV charge circuit 104 is transferring energy, such as from the energy supply 106, to actively charge the CSD 102. In some embodiments, the below method can be accomplished simultaneously while the capacitor reforming task is accomplished.

At 404, the HV monitoring circuit 110 collects voltage level measurements across the CSD 102 as the HV charge circuit 104 charges the CSD 102. The voltage level measurements can be taken at regular intervals to form a charge time profile (not shown).

At 406, the one or more processors analyze voltage level measurements to determine a charge time to a predetermined voltage level, which in some embodiments can be the predetermined voltage level 308 of FIG. 3. In general, the charge time to the predetermined voltage level 308 will increase over time due to increases of the internal battery resistance (e.g., the R_{ESR} 108 of the energy supply 106 (FIG. 1)). In some embodiments, the charge time to reach the predetermined voltage level 308 for an IMD that is nearing its end of life can be in a range of approximately 28-33 seconds primarily due to the increase in R_{ESR} 108 and/or other loss of energy within the energy supply 106 and/or IMD.

At 408, the one or more processors determine an adjusted charge time to the predetermined voltage level 308 by removing the battery voltage/ R_{ESR} 108 effect. These factors from IMD components outside of the CSD 102 can be determined through other processes, such as when monitoring the health of the battery (e.g., energy supply 106).

At 410, the one or more processors compare the adjusted charge time to the baseline charge time that was determined and stored e.g., according to the method of FIG. 2.

At 412, the one or more processors determine whether the adjusted charge time is within a tolerance or threshold range of the baseline charge time. If the adjusted charge time is not within the tolerance, at 414 the one or more processors declare the associated charge time to be a failure signature and can initiate a warning to advise that the CSD 102 has a fault or is in a failure state, such as by transmitting a communication to inform the patient and/or health care professionals of the failure state. The failure state can indicate a potential to fail, or a partial failure, and can be used together with other data, such as other logged failure states over time, to determine that the health of the CSD 102 is beginning to erode and/or may be at a level that service or replacement of the ICD may be considered.

If at 412 the adjusted charge time is within the tolerance, the CSD 102 is in a normal or non-failure state. At 416, the one or more processors store the adjusted charge time and/or the non-adjusted charge time (determined at 406) to allow monitoring of charge time performance over time (e.g., over years). In some embodiments, the associated charge profile(s) (not shown) can also be stored. In some embodiments, the adjusted charge time and/or charge profile can be transmitted to other devices.

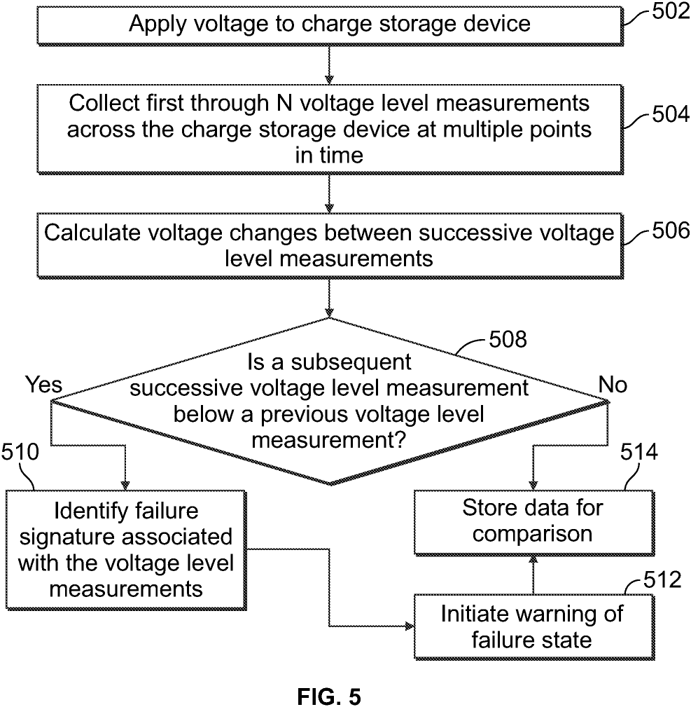

FIG. 5 illustrates a method for measuring a continuity of capacitor voltage of the CSD 102 (FIG. 1) during a charging task in accordance with embodiments herein. The operations of FIG. 5 may be implemented by hardware, firmware, circuitry and/or one or more processors housed partially and/or entirely within an IMD, a local external device, remote server or more generally within a healthcare system. Optionally, the operations of FIG. 5 may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors. For example, the continuity of measurements, such as along a charge profile, can be used to indicate a normal state or a failure state of the CSD 102. Because a failure state may occur over a short period of time, many successive measurements that are separated by short time intervals are collected.

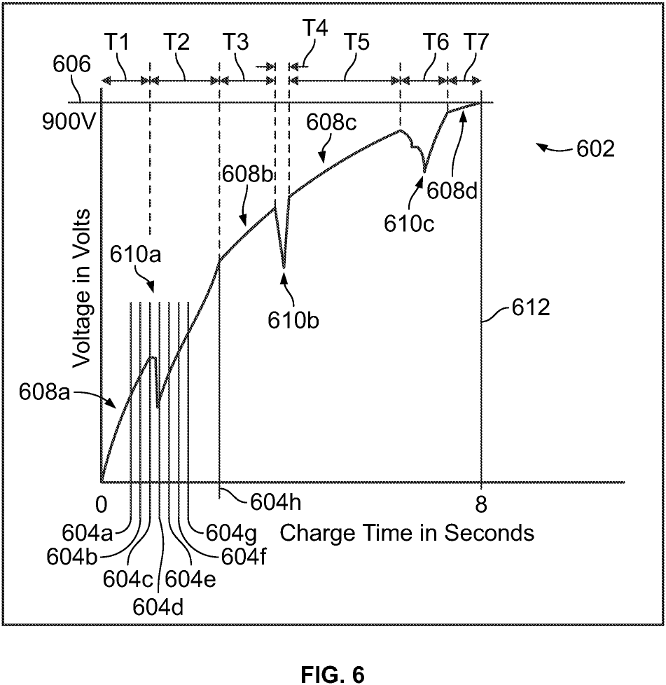

FIG. 6 illustrates a charge profile 602 that has multiple failure states associated with voltage drops (e.g., scintillation voltage drops) in accordance with embodiments herein. The horizontal axis plots charge time of the CSD 102 (FIG. 1) in seconds, while the vertical axis plots the voltage of the CSD 102 in volts. By way of example only, charge time 612 can be 8 seconds. It is recognized that the voltages, times, and/or shapes of the charge profile illustrated in FIG. 6 are examples and are not limiting. FIGS. 5 and 6 will be discussed together.

In some embodiments, scintillations (e.g., partial discharges) take power out of the energy supply 106 and an increasing number of scintillations are often accompanied by an increase in leakage current. Scintillation voltage drops can be caused by dielectric punch-through (e.g., short circuit between adjacent dielectric layers), and/or corrosion that can be, for example, a result of chlorine, bromine and/or iodine contamination (e.g., group 7 halogens). If not addressed, this contamination can cause decay of the CSD 102, reducing usage lifespan (e.g., from 10 years to 6-7 years). Another source of undesirable changes in voltage over time (DV/DT) wherein the voltage drops during a charging task can stem from using non-film formers (e.g., iron, copper, etc.) in the oxide forms on the aluminum. The presence of the non-film formers, in contrast with aluminum oxide, will negatively affect the charge time. In some embodiments, a negative DV/DT that exceeds a predetermined threshold can indicate loss of energy of the system as a whole.

In general, the “healing” of the capacitor(s) that takes place during the reforming task can take extra energy in contrast with simply charging the capacitor(s). For example, a self-healing event that occurred from 390 V to 400 V can require two extra joules to charge the capacitor, and thus drains power from the battery. If contamination or corrosion exists, such as iron contamination and/or chlorine/chloride contamination, these issues can return and cause contamination/corrosion in the future.

At 502, the one or more processors apply voltage to charge the CSD 102 in a charging operation. In some cases, the HV charge circuit 104 is actively charging the CSD 102 by transferring energy from the energy supply 106 to the CSD 102. In some embodiments, the method of FIG. 5 can occur during the reforming task or on demand.

At 504, the HV monitoring circuit 110 collects N voltage level measurements 604 (FIG. 6) across the CSD 102 at multiple points in time during the charging operation to generate a charge profile. For example, the voltage level measurements 604 can be collected periodically or separated by short time intervals until the predetermined voltage level 606 is reached. Not all of the voltage level measurements 604 are shown for clarity.

At 506, the one or more processors calculate voltage changes between successive voltage level measurements 604. For example, a change in voltage can be calculated between the voltage level measurements 604a and 604b, the voltage level measurements 604b and 604c, the voltage level measurements 604c and 604d, and so on.

At 508, the one or more processors determine whether a subsequent successive voltage level measurement 604 is below a previous voltage level measurement. In some embodiments, the capacitor health monitor 112 or other processor(s) can monitor the slope of the voltage curve (DV/DT) of the charge profile 602 to determine if the slope is not positive (e.g., unchanged, negative). For example, the one or more processors can determine whether the rate of change of the charge profile 602 has a negative slope between successive measurements. In some embodiments, a decrease in a successive voltage level measurement 604 can indicate corrosion and/or contamination. The voltage change between the voltage level measurements 604a and 604b and the voltage level measurements 604b and 604c show increases in voltage during which the CSD 102 is actively being charged. This segment of the charge profile 602 over time period T1 can be a monotonically increasing segment 608a. However, the voltage change between the voltage level measurements 604c and 604d show a decrease in voltage during which the CSD 102 is actively being charged. This segment of the charge profile 602 included within time period T2 can be a failure signature 610a. In some embodiments, the decrease in voltage, slope of the voltage curve, etc., can be compared to a threshold to identify changes that exceed the threshold, which can be identified as failure signatures.

If the decrease in voltage is determined at 508 (e.g., slope of DV/DT decreased), at 510, the one or more processors identify the failure signature 610a in the charge profile 602. The failure signature 610a can represent a partial discharge. The term “partial discharge”, when used to describe a state or condition of a CSD 102, shall mean that the voltage level of the CSD 102 during an active charging task decreased to a level greater than zero volts (e.g., not fully discharged). A partial discharge can result from a dielectric punch-through (e.g., partial bridge of adjacent layers of a capacitor within the charge storage device) or from contamination. The terms “recover” and “recovery”, when used to describe a state or condition of a CSD 102, shall mean that the voltage level of the CSD 102 during an active charging task and immediately subsequent to a partial discharge, increases to a projected/expected level of the associated charge profile. For example, the voltage drop can be a one-time event that is healed by the reforming task. In other embodiments the voltage drop can indicate a more catastrophic event, such as a loss of a layer of a capacitor within the CSD 102.

In some embodiments the partial discharge can be followed by a recovery in the voltage level measurements 604. During time period T2, the partial discharge is represented as a decrease in the charge profile 602, such as between the voltage level measurements 604c and 604d. The charge profile 602 subsequently recovered and increased to resume a monotonic charge profile, all while the CSD 102 is being actively charged. For example, the voltage change between successive ones of the voltage level measurements 604d, 604e, 604f, 604g show increasing voltage levels through the recovery. The recovery extends to the voltage level measurement 604h and the start of the time period T3, where the charge profile 602 recovers to the projected/expected level.

Although not shown, voltage level measurements 604 can be compared to identify monotonically increasing segments 608b, 608c, 608d over time periods T3, T5, T7, respectively, and failure signatures 610b, 610c over time periods T4, T6, respectively. The failure signatures 610 can have different decreases over different lengths of time, followed by increases in voltage over different lengths of time. In some embodiments, even though the CSD 102 continues to charge and exhibits a “normal” charge curve, damage has occurred in the capacitor(s). The damage may be a chronic problem (e.g., a loss of a layer of a capacitor) or may be a one-time event, either of which can result in a longer charge time that is not due to the effects of R_{ESR} 108 or other losses within the IMD.

At 512, the one or more processors can initiate a warning to notify the patient and/or health care professionals of a failure state. In some embodiments, a warning can be initiated after the one or more processors determine whether the failure signature(s) 610 meet or exceed certain thresholds. In some embodiments the warning can be a communication that is output or transmit to indicate that one or more of the capacitors of the CSD 102 is experiencing the failure state.

At 514, the one or more processors store data associated with charge profile 602 and any identified failure signatures 610 to allow monitoring of charging performance over time. The data can be stored within the IMD as well as transmitted to other computers, systems, and the like.

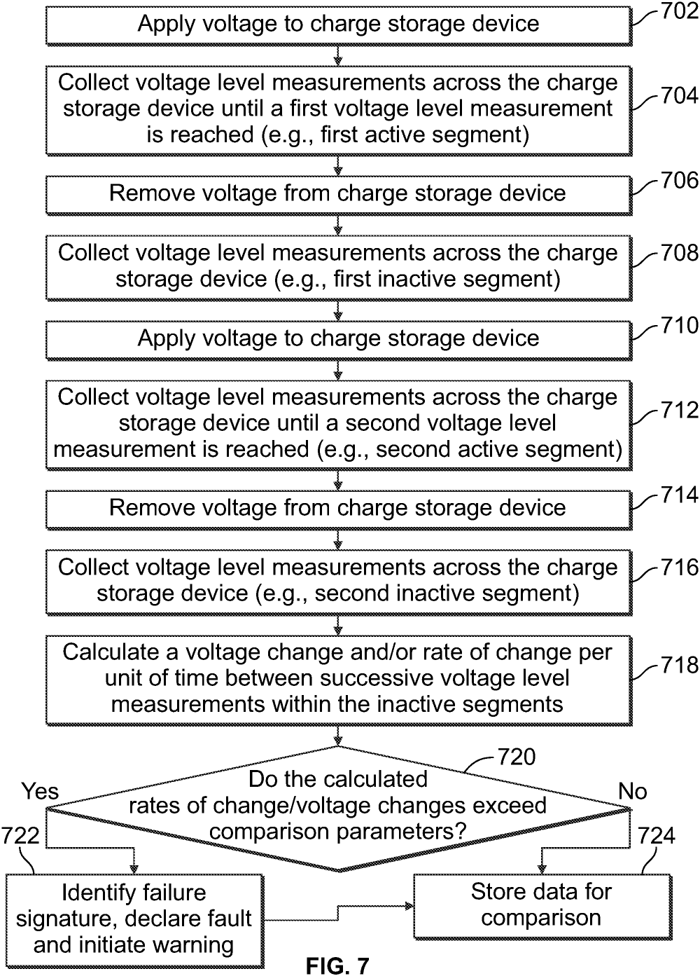

FIG. 7 illustrates a method for measuring health of the CSD 102 (FIG. 1) by measuring leakage (e.g., voltage drop, leakage current, etc.) during a charging task in accordance with embodiments herein. The operations of FIG. 7 may be implemented by hardware, firmware, circuitry and/or one or more processors housed partially and/or entirely within an IMD, a local external device, remote server or more generally within a healthcare system. Optionally, the operations of FIG. 7 may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors. As with the method of FIG. 5, the continuity of voltage level measurements, such as along a charge profile, can be used to indicate a normal state or a failure state of the CSD 102 and many successive voltage level measurements that are separated by short time intervals can be collected. In some embodiments, the method of FIG. 7 can occur during the reforming task, on a schedule, or on demand while the IMD is implanted in the patient, and can also occur in advance of implanting the IMD to ensure proper operation and to establish a baseline.

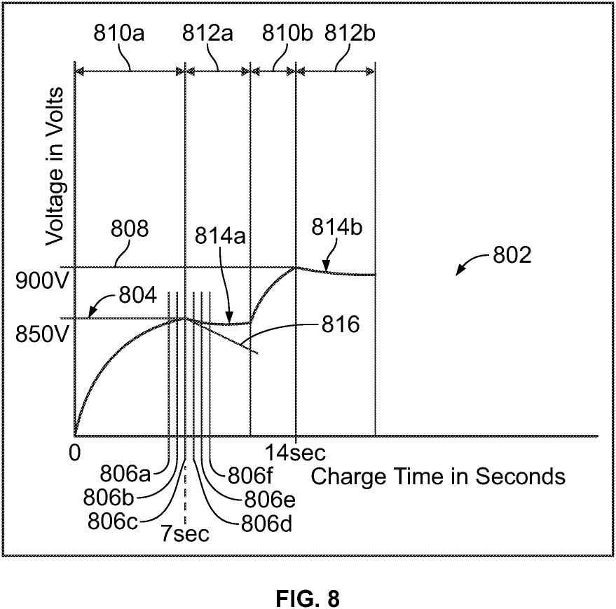

FIG. 8 illustrates a charge profile 802 wherein the CSD 102 (FIG. 1) is charged several successive times with decay periods therebetween during which the CSD 102 is not being charged in accordance with embodiments herein. The horizontal axis plots charge time of the CSD 102 in seconds, while the vertical axis plots the voltage of the CSD 102 in volts. In general, an increase in the rise over run bleed rate in the decay periods compared to a baseline can indicate that a partial short exists or chemistry may have changed in the CSD 102. FIGS. 7 and 8 will be discussed together.

At 702, the one or more processors apply voltage to charge the CSD 102 in a charging operation to a first predetermined voltage level 804 (indicated with a horizontal solid line), such as 850 V.

At 704, the HV monitoring circuit 110 collects N voltage level measurements 806 across the CSD 102 at multiple points in time during the first charging operation to generate the charge profile 802 (not all of the voltage level measurements 806 are shown for clarity). For example, the first voltage level measurements 806a, 806b can be collected periodically or separated by short time intervals until the first predetermined voltage level 804 is reached at the voltage level measurement 806c, such as at 7 seconds. The time while the voltage is applied to charge the CSD 102 to the first predetermined voltage level 804 is a first active segment 810a of the charge profile 802. As used herein, an “active segment” is a period of time wherein the CSD 102 is being charged.

At 706, the one or more processors remove (e.g., electrically decouple) the voltage from the CSD 102.

At 708, the HV monitoring circuit 110 continues to collect voltage level measurements 806d, 806e, 806f across the CSD 102 at multiple points in time during a first inactive segment 812a. As used herein, an “inactive segment” 812 is a period of time wherein the CSD 102 is not being charged. During the inactive segment 812 the CSD 102 is experiencing leakage and the voltage level measurements 806 collected across the CSD 102 decrease.

At 710, the one or more processors apply voltage to charge the CSD 102 to a second predetermined voltage level 808 in another charging operation, such as a second active segment 810b.

At 712, the HV monitoring circuit 110 collects the voltage level measurements 806 (not shown) across the CSD 102 at multiple points in time during the second charging operation. For example, the voltage level measurements 806 can be collected periodically or separated by short time intervals until the second predetermined voltage level 808 is reached, such as 900 V, such as at 14 seconds. The time while the voltage is applied to charge the CSD 102 is a second active segment 810b of the charge profile 802. In some embodiments, the second predetermined voltage level 808 is higher than the first predetermined voltage level.

At 714, the one or more processors remove the voltage from the CSD 102. In some embodiments, if additional charging operations are to be accomplished, the method can return to 710.

At 716, the HV monitoring circuit 110 collects voltage level measurements 806 (not shown) across the CSD 102 at multiple points in time during a second inactive segment 812b. Monitoring of the second inactive segment 812b can continue for a predetermined amount of time or until the CSD 102 decays to a predetermined voltage level.

At 718, the one or more processors calculate voltage changes and/or determine a rate of change per unit of time between successive voltage level measurements 806 that were collected within the first inactive segment 812a and the second inactive segment 812b.

At 720, the one or more processors determine whether any of the calculated rates of change or voltage level changes exceed a leakage profile or a threshold, and if so, determine a failure state. In some embodiments, if this charging task is a baseline charging task, the charge profile 802 can include one or more baseline leakage profiles 814a, 814b (e.g., normal decay) extending through the inactive segments 812a, 812b. For example, a baseline leakage profile 814 can be determined and stored before the IMD is implanted within the patient. After time has elapsed (e.g., storage of device or device is implanted in the patient), the calculated leakage parameters (of 718) can be compared to the baseline leakage profile 814. In other embodiments, the calculated leakage parameters can be compared to an average leakage profile, leakage profiles indicative of a healthy CSD 102 and/or failure state(s) of charge storage device(s) 102, and the like. In some embodiments, if a calculated rate of change, indicated as line 816 extending downward (e.g., negative slope), exceeds a threshold rate of change and/or a leakage profile, a failure state is determined.

At 722, the one or more processors declare a failure signature within the inactive segment 812 and can initiate a warning to advise that the CSD 102 has a fault or is in a failure state, such as by transmitting a communication to inform the patient and/or health care professionals of the failure state.

At 724, the one or more processors store data associated with charge profile 802, leakage data, and/or any identified failure signatures to allow monitoring of leakage performance over time (e.g., over years). As discussed above, storage can be local within the IMD or transmitted to another device or system.

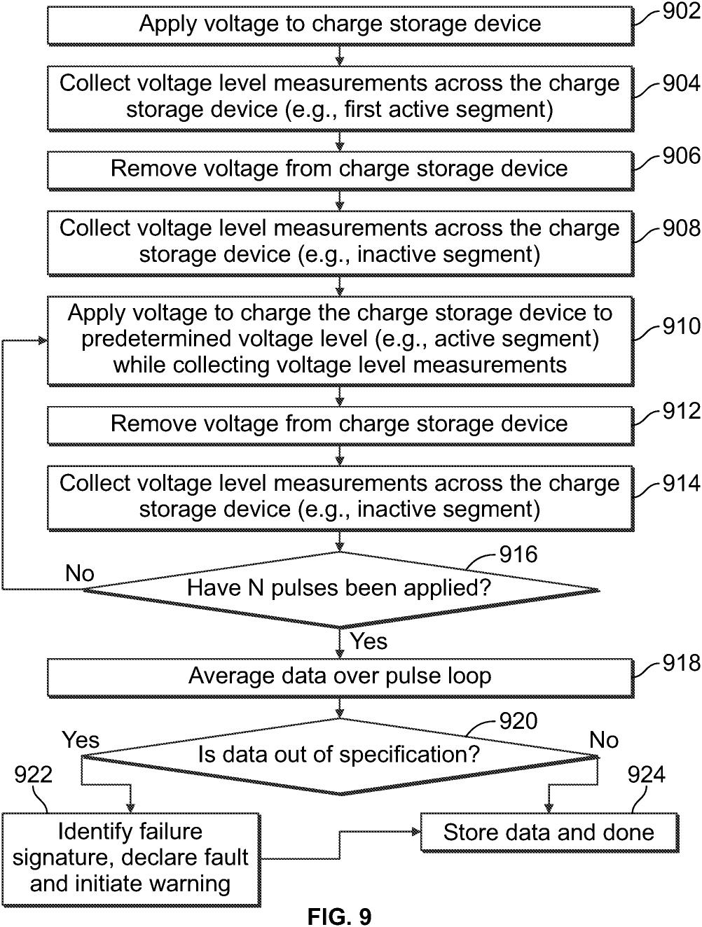

FIG. 9 illustrates a method for measuring health of the CSD 102 (FIG. 1) by measuring micro-decay during a pulsed charging task at high voltage in accordance with embodiments herein. The operations of FIG. 9 may be implemented by hardware, firmware, circuitry and/or one or more processors housed partially and/or entirely within an IMD, a local external device, remote server or more generally within a healthcare system. Optionally, the operations of FIG. 9 may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors. In some embodiments, the leakage currents associated with the pulses can be evaluated. As with the above methods, the continuity of voltage level measurements, such as along a charge profile, can indicate a normal state or a failure state of the CSD 102 and many successive measurements that are separated by short time intervals can be collected. The method of FIG. 9 can run simultaneously with the methods of FIGS. 5 and 7, the reforming task, etc.

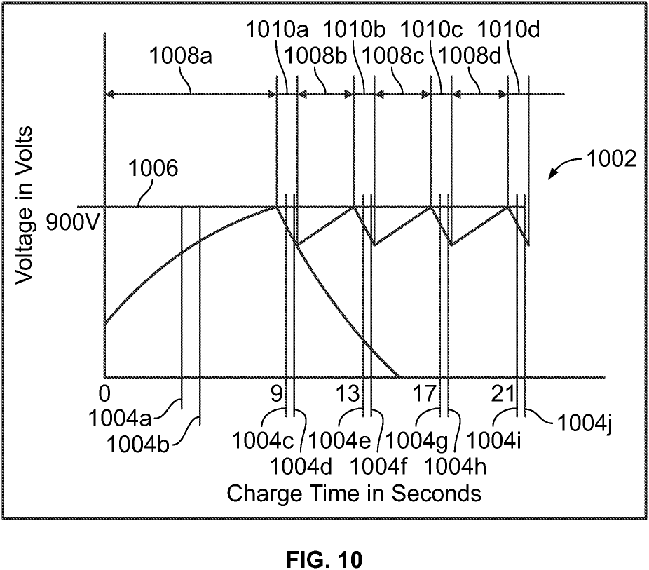

FIG. 10 illustrates a charge profile 1002 wherein a series of quick pulses at peak voltage is applied to the CSD 102 (FIG. 1) in accordance with embodiments herein. A series of pulse loops is formed, wherein each pulse loop includes an active segment and an inactive segment. Each pulse loop can be, for example, 4 seconds. The horizontal axis plots charge time of the CSD 102 (FIG. 1) in seconds, while the vertical axis plots the voltage of the CSD 102 in volts. FIGS. 9 and 10 will be discussed together.

At 902, the one or more processors apply voltage to charge the CSD 102 in a charging operation.

At 904, the HV monitoring circuit 110 collects N voltage level measurements 1004a, 1004b (for clarity, not all of the voltage level measurements 1004 are shown in FIG. 10) across the CSD 102 at multiple points in time during a first charging operation to generate the charge profile 1002. For example, the voltage level measurements 1004 can be collected periodically or separated by short time intervals throughout the method. The voltage is applied until a predetermined voltage level 1006 is reached. In some embodiments the predetermined voltage level 1006 can be a peak voltage level, such as 850V, 900V, etc., but the embodiments are not so limited. The time while the voltage is applied to charge the CSD 102 to the predetermined voltage level 1006 is an active segment 1008a of the charge profile 1002.

At 906, the one or more processors remove (e.g., electrically decouple) the voltage from the CSD 102.

At 908, the HV monitoring circuit 110 collects voltage level measurements 1004c, 1004d across the CSD 102 at multiple points in time during an inactive segment 1010a. The CSD 102 experiences leakage or decay during the inactive segment 1010a, and the voltage level measured across the CSD 102 decreases. The inactive segment 1010a can continue for a predetermined amount of time (e.g., 1 second, 2 seconds, 10 seconds, etc.) or until the CSD 102 decays to a predetermined voltage level (not indicated).

At 910, the one or more processors apply voltage to charge the CSD 102 to the predetermined voltage level 1006 in a charging operation (active segment 1008b) while collecting the voltage level measurements 1004 (not shown) across the CSD 102. The CSD 102 can be charged until the predetermined voltage level 1006 is reached. In other embodiments, the CSD 102 can be charged for a predetermined length of time.

At 912, the one or more processors remove the voltage from the CSD 102.

At 914, the HV monitoring circuit 110 collects voltage level measurements 1004e, 1004f across the charge storage device during inactive segment 1010b. The inactive segment 1010b can continue for a predetermined amount of time or until the CSD 102 decays to a predetermined voltage level.

At 916, the one or more processors determine whether N pulses have been applied. In some embodiments, N pulses (e.g., 5 pulses, 10 pulses, 20 pulses, 100 pulses, a range of pulses from 10-100 pulses, etc.) forming active segments 1008 can be applied followed by an equal number of decay or inactive segments 1010. By way of example only, three pulses are shown in FIG. 10, including active segments 1008b, 1008c, 1008d and inactive segments 1010b, 1010c, 1010d. In some embodiments, each of the inactive segments 1010 can have the same duration of time as each other and the active segments 1008 can have the same duration of time as each other.

If more pulses are to be applied, the method returns to 910. If N pulses have been applied, at 918, the one or more processors average the data associated with the pulse loops. For example, charge data (e.g., voltage level measurements acquired over the active segments 1008) can be averaged and decay data (e.g., voltage level measurements acquired over the inactive segments 1010) can be averaged. In some embodiments, the voltage level measurements 1004e, 1004g, 1004i collected at the same time within different inactive segments 1010b, 1010c, 1010d can be averaged. These calculations can increase the accuracy of leakage extraction.

At 920, the one or more processors determine if the collected data is out of specification. Several different comparisons can be made. In some embodiments, if the data is acquired before the IMD is implanted in a patient, the data is baseline data. The charge rate and/or decay rate can be compared to data that represents the CSD 102 at its beginning of life, which may be, for example, different than specification data of the CSD 102 after 5 or 7 years. In other embodiments, if the data is acquired after the IMD is implanted in a patient, the data (e.g., charge rate, decay rate, etc., and/or a variance of the average data) can be compared to prior and/or baseline data to determine if the data is out of specification.

If the data is out of specification, at 922, the one or more processors identify a failure signature associated with at least one of the tested parameters, declare a fault, and/or can initiate a warning to advise that the CSD 102 has a fault or is in a failure state, such as by transmitting a communication to inform the patient and/or health care professionals of the failure state.

At 924, the one or more processors store the data. The data can be used for future comparisons within the IMD, sent to remote systems, used to track trends associated with a plurality of charge storage devices 102 and IMDs, etc.

Technical advantages of several embodiments herein include the acquisition of solid baseline data with more certainty of response at the most extreme voltages. The data acquired over the series of micro-pulses (e.g., the repeated active segments 1008 and inactive segments 1010) can provide information to determine how reformed the capacitor(s) within the CSD 102 are. The methods and devices further provide the technical benefit of monitoring the performance and capability of the CSD 102 with better accuracy over time (e.g., months, years), which improves the ability to determine a life expectancy of a particular implanted medical device. Further, the methods of applying micro-pulses, such as at high voltage, can further repair the electrochemistry of the capacitor(s) (e.g., reforming) within the CSD 102, prolonging the life and/or reliability of the device. In addition, the application of short pulses at high voltage can provide another method of monitoring the health of the CSD 102 without putting unnecessary stress on the energy supply 106.

### Implantable Medical Device

FIG. 11 shows an exemplary IMD 1100 that is implanted into the patient as part of an implantable cardiac system. The IMD 1100 has a housing 1101 to hold the electronic/computing components. The housing 1101 (which is often referred to as the “can”, “case”, “encasing”, or “case electrode”) may be programmably selected to act as the return electrode for certain stimulus modes. Housing 1101 further includes a connector (not shown) with a plurality of terminals 1102, 1104, 1106, 1108, and 1110. The terminals may be connected to electrodes that are located directly on the housing of the IMD 1100 and/or connected to one or more leads that are located at various locations within and about the heart. The type and location of each electrode may vary. The IMD 1100 includes a programmable microcontroller 1120 that controls various operations of the IMD 1100, including cardiac monitoring, monitoring of the health of the CSD 102 (FIG. 1), and stimulation therapy. Microcontroller 1120 includes a microprocessor (or equivalent control circuitry), RAM and/or ROM memory, logic and timing circuitry, state machine circuitry, and I/O circuitry.

The IMD 1100 includes a high voltage monitoring circuit 1174 configured to measure the voltage across a shocking circuit 1180 as explained herein. The high voltage monitoring circuit 1174 connects to the microcontroller 1120 and the shocking circuit 1180. The monitoring circuit 1174 is configured to collect voltage level measurements across the shocking circuit 1180 (e.g., charge storage device) at multiple points in time during the charging operation.

The microcontroller 1120 includes a capacitor health monitor 1132 and other logic to perform the methods described herein. When executing a charging operation, the microcontroller 1120 can direct the battery 1172 and a high voltage charge circuit to charge the shocking circuit 1180. The shocking circuit 1180 can include one or more capacitors (e.g., charge storage device). When executing the capacitor health monitor 1132, the microcontroller 1120 collects voltage level measurements across the charge storage device to form a charge profile. In some embodiments, the microcontroller 1120 can collect the voltage level measurements via the monitoring circuit 1174 that is in turn collecting the voltage level measurements across the shocking circuit 1180. The charge profile can exhibit voltage level changes across the charge storage device over a charge storage device charging operation. When executing the capacitor health monitor 1132, the microcontroller 1120 analyzes the voltage level measurements to identify a failure signature in the charge profile, and based thereon, the microcontroller 1120 generates an output indicative of a failure state for the charge storage device. The microcontroller 1120 can determine, based on at least the voltage level measurements that are collected over various charging tasks of the IMD, whether a fault, fault condition, failure signature, failure state, and the like are occurring or likely to occur within the charge storage device. For example, the failure state for one or more capacitors of the charge storage device can include at least one of i) a loss of a dielectric layer of a capacitor, ii) corrosion detected, iii) oxide formation, iv) at least one of iron, chlorine, bromine, iodine, or copper contamination, or v) short circuit between adjacent dielectric layers.

When executing the capacitor health monitor 1132, the microcontroller 1120 can identify that the failure signature that occurred while actively charging the charge storage device represents a partial discharge in the charge profile that is expressed by a decrease in the voltage level measurements, followed by a recovery in the voltage level measurements. Further, the microcontroller can identify that when the charge storage device is in a non-failure state, the charge profile exhibits a voltage level that monotonically increases throughout a period of time in which the charge storage device is actively being charged, and when the charge storage device is in a failure state, the charge profile exhibits one or more voltage drops during the period of time in which the charge storage device is actively being charged.

When analyzing the voltage level measurements, the microcontroller 1120, when executing the capacitor health monitor 1132, can calculate a rate of change per unit of time in the charge profile and determine when the rate of change has a negative slope. Further, when analyzing the voltage level measurements, the microcontroller 1120 can calculate voltage changes between successive first and second voltage level measurements and determine when the second voltage level measurement is below the first voltage level measurement.

When executing the capacitor health monitor 1132, the microcontroller 1120 can identify that the charge profile further includes an active segment and an inactive segment. The active segment can correspond to a period of time during the charging operation in which the charge storage device is actively being charged, and the inactive segment corresponds to a period of time during the charging operation in which the charge storage device is not being charged.

The IMD 1100 further includes a pulse generator 1122 that generates stimulation pulses for delivery by one or more electrodes coupled thereto. The pulse generator 1122 is controlled by the microcontroller 1120 via control signal 1124. The pulse generator 1122 is coupled to the select electrode(s) via an electrode configuration switch 1126, which includes multiple switches for connecting the desired electrodes to the appropriate I/O circuits, thereby facilitating electrode programmability. The switch 1126 is controlled by a control signal 1128 from the microcontroller 1120. In the example of FIG. 11, a single pulse generator 1122 is illustrated. Optionally, the IMD 1100 may include multiple pulse generators, similar to pulse generator 1122, where each pulse generator is coupled to one or more electrodes and controlled by the microcontroller 1120 to deliver select stimulus pulse(s) to the corresponding one or more electrodes.

The microcontroller 1120 includes various modules to implement the functionality of the IMD 1100. For example, the microcontroller 1120 controls the timing of the stimulation pulses (e.g., pacing rate, atrio-ventricular (AV) delay, atrial interconduction (A-A) delay, or ventricular interconduction (V-V) delay, etc.). Microcontroller 1120 detects arrhythmia conditions and may review and analyze one or more features of the morphology of cardiac signals. Although not shown, the microcontroller 1120 may further include other dedicated circuitry and/or firmware/software components that assist in monitoring various conditions of the patient’s heart and managing pacing therapies.

The IMD 1100 is further equipped with a communication modem (modulator/demodulator) 1140 to enable wireless communication with other devices, implanted devices and/or external devices. In one implementation, the communication modem 1140 may use high frequency modulation of a signal transmitted between a pair of electrodes. As one example, the signals may be transmitted in a high frequency range as such signals travel through the body tissue and fluids without stimulating the heart or being felt by the patient. The communication modem 1140 may be implemented in hardware as part of the microcontroller 1120, or as software/firmware instructions programmed into and executed by the microcontroller 1120. Alternatively, the modem 1140 may reside separately from the microcontroller 1120 as a standalone component.

The IMD 1100 includes sensing circuitry 1144 selectively coupled to one or more electrodes that perform sensing operations, through the switch 1126 to detect the presence of cardiac activity in the right chambers of the heart. The sensing circuitry 1144 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the IMD 1100 to sense low amplitude signal characteristics of atrial fibrillation. Switch 1126 determines the sensing polarity of the cardiac signal by selectively closing the appropriate switches. In this way, the clinician may program the sensing polarity independent of the stimulation polarity.

The output of the sensing circuitry 1144 is connected to the microcontroller 1120 which, in turn, triggers or inhibits the pulse generator 1122 in response to the absence or presence of cardiac activity. The sensing circuitry 1144 receives a control signal 1146 from the microcontroller 1120 for purposes of controlling the gain, threshold, polarization charge removal circuitry (not shown), and the timing of any blocking circuitry (not shown) coupled to the inputs of the sensing circuitry.

In the example of FIG. 11, a single sensing circuit 1144 is illustrated. Optionally, the IMD 1100 may include multiple sensing circuits, similar to sensing circuit 1144, where each sensing circuit is coupled to one or more electrodes and controlled by the microcontroller 1120 to sense electrical activity detected at the corresponding one or more electrodes. The sensing circuit 1144 may operate in a unipolar sensing configuration or in a bipolar sensing configuration.

The IMD 1100 further includes an analog-to-digital (A/D) data acquisition system (DAS) 1150 coupled to one or more electrodes via the switch 1126 to sample cardiac signals across any pair of desired electrodes. The data acquisition system 1150 is configured to acquire intracardiac electrogram signals, convert the raw analog data into digital data, and store the digital data for later processing and/or telemetric transmission to an external device 1154 (e.g., a programmer, local transceiver, or a diagnostic system analyzer). The data acquisition system 1150 is controlled by a control signal 1156 from the microcontroller 1120.

The microcontroller 1120 is coupled to a memory 1160 by a suitable data/address bus 1162. The programmable operating parameters used by the microcontroller 1120 are stored in memory 1160 and used to customize the operation of the IMD 1100 to suit the needs of a particular patient. The operating parameters of the IMD 1100 may be non-invasively programmed into the memory 1160 through a telemetry circuit 1164 in telemetric communication via communication link 1166 with the external device 1154. The telemetry circuit 1164 allows intracardiac electrograms and status information relating to the operation of the IMD 1100 (as contained in the microcontroller 1120 or memory 1160) to be sent to the external device 1154 through the established communication link 1166. For example, the communication link 1166 can indicate that one or more capacitors of the charge storage device (e.g., within the shocking circuit 1180) is experiencing a failure state. In some embodiments, the external device 1154 can initiate any of the processes disclosed herein, and electronics within the external device 1154 can provide processing, such as to analyze the voltage level measurements, identify failure signature(s), generate output indicative of a failure state for the charge storage device, and the like.

The IMD 1100 can further include one or more physiologic sensors 1170. Such sensors are commonly referred to as “rate-responsive” sensors because they are typically used to adjust pacing stimulation rates according to the exercise state of the patient.

The battery 1172 (e.g., energy supply) provides operating power to all of the components in the IMD 1100 and can be housed within the IMD 1100. The battery 1172 is capable of operating at low current drains for long periods of time, and is capable of providing high-current pulses (e.g., for capacitor charging) when the patient requires a shock pulse (e.g., in excess of 2 A, at voltages above 2 V, for periods of 120 seconds or more). The battery 1172 is configured to charge the charge storage device during a charging operation. The battery 1172 also desirably has a predictable discharge characteristic so that elective replacement time can be detected. As one example, the IMD 1100 employs lithium/silver vanadium oxide batteries, but is not so limited.

The IMD 1100 may be operated as an implantable cardioverter/defibrillator (ICD) device, which detects the occurrence of an arrhythmia and automatically applies an appropriate electrical shock therapy to the heart aimed at terminating the detected arrhythmia. To this end, the microcontroller 1120 further controls a shocking circuit 1180 by way of a control signal 1182. The shocking circuit 1180 generates shocking pulses of low (e.g., up to 0.5 joules), moderate (e.g., 0.5-10 joules), or high energy (e.g., 11 to 40 joules), as controlled by the microcontroller 1120. Such shocking pulses are applied to the patient’s heart through shocking electrodes.

### External Device

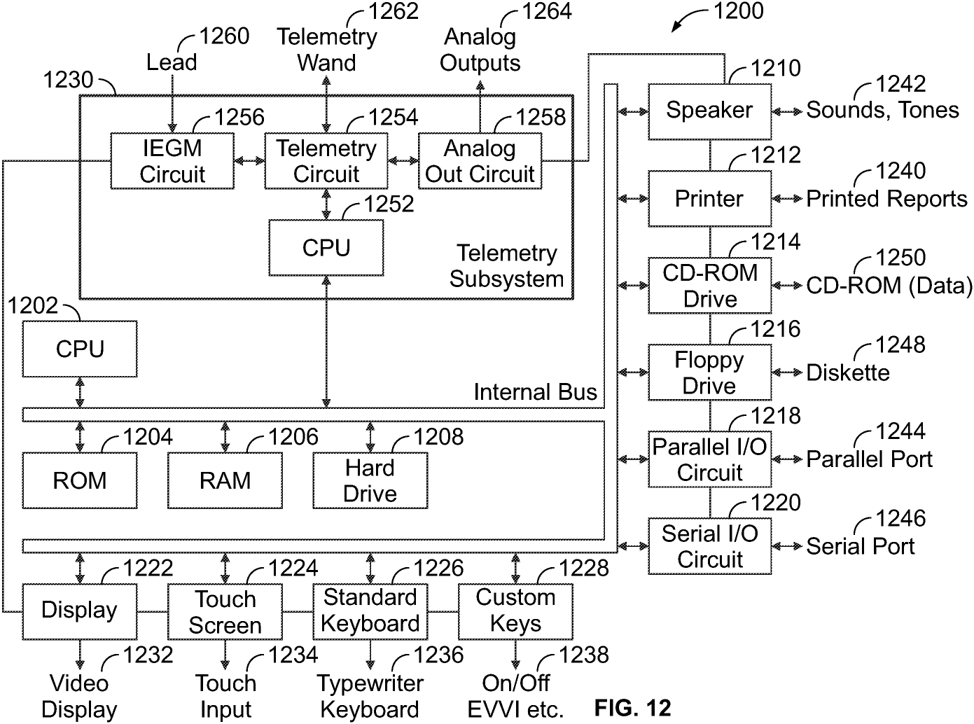

FIG. 12 illustrates a functional block diagram of an external device 1200 that is operated in accordance with the processes described herein and to interface with implantable medical devices as described herein. The external device 1200 may be a workstation, a portable computer, an IMD programmer, a PDA, a cell phone and the like. The external device 1200 includes an internal bus that connects/interfaces with a Central Processing Unit (CPU) 1202, ROM 1204, RAM 1206, a hard drive 1208, the speaker 1210, a printer 1212, a CD-ROM drive 1214, a floppy drive 1216, a parallel I/O circuit 1218, a serial I/O circuit 1220, the display 1222, a touch screen 1224, a standard keyboard connection 1226, custom keys 1228, and a telemetry subsystem 1230. The internal bus is an address/data bus that transfers information between the various components described herein. The hard drive 1208 may store operational programs as well as data, such as waveform templates and detection thresholds.

The CPU 1202 typically includes a microprocessor, a microcontroller, or equivalent control circuitry, designed specifically to control interfacing with the external device 1200 and with the IMD. The CPU 1202 performs the processes discussed above. For example, the CPU 1202 may perform all or a portion of the determinations of failure signatures and failure states, as well as the comparisons of collected voltage level measurements to baseline parameters and/or previously acquired voltage level measurements and/or other data to determine whether early failure conditions exist. The CPU 1202 can also initiate warnings based on the various methods herein.

The CPU 1202 may include RAM or ROM memory, logic and timing circuitry, state machine circuitry, and I/O circuitry to interface with the IMD. The display 1222 (e.g., may be connected to the video display 1232). The touch screen 1224 may display graphic information relating to the IMD. The display 1222 displays various information related to the processes described herein. The touch screen 1224 accepts a user’s touch input 1234 when selections are made. The keyboard 1226 (e.g., a typewriter keyboard 1236) allows the user to enter data to the displayed fields, as well as interface with the telemetry subsystem 1230. Furthermore, custom keys 1228 turn on/off 1238 (e.g., EVVI) the external device 1200. The printer 1212 prints copies of reports 1240 for a physician to review or to be placed in a patient file, and speaker 1210 provides an audible warning (e.g., sounds and tones 1242) to the user. The parallel I/O circuit 1218 interfaces with a parallel port 1244. The serial I/O circuit 1220 interfaces with a serial port 1246. The floppy drive 1216 accepts diskettes 1248. Optionally, the floppy drive 1216 may include a USB port or other interface capable of communicating with a USB device such as a memory stick. The CD-ROM drive 1214 accepts CD ROMs 1250.

The telemetry subsystem 1230 includes a central processing unit (CPU) 1252 in electrical communication with a telemetry circuit 1254, which communicates with both an intracardiac electrogram (IEGM) circuit 1256 and an analog out circuit 1258. The circuit 1256 may be connected to leads 1260. The circuit 1256 is also connected to the implantable leads to receive and process IEGM cardiac signals as discussed above. Optionally, the IEGM cardiac signals sensed by the leads may be collected by the IMD and then transmitted, to the external device 1200, wirelessly to the telemetry subsystem 1230 input.

The telemetry circuit 1254 is connected to a telemetry wand 1262. The analog out circuit 1258 includes communication circuits to communicate with analog outputs 1264. The external device 1200 may wirelessly communicate with the IMD and utilize protocols, such as Bluetooth, GSM, infrared wireless LANs, HIPERLAN, 3G, satellite, as well as circuit and packet data protocols, and the like. Alternatively, a hard-wired connection may be used to connect the external device 1200 to the IMD.

### Alternative Embodiments

Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a subcutaneous cardioverter defibrillator, cardiac monitoring device, pacemaker, cardioverter, cardiac rhythm management device, defibrillator, neurostimulator, leadless monitoring device, leadless pacemaker, and the like. The IMD may measure electrical and/or mechanical information. For example, the IMD may include one or more structural and/or functional aspects of the device(s) described in U.S. Pat. No. 9,333,351, entitled “NEUROSTIMULATION METHOD AND SYSTEM TO TREAT APNEA” issued May 10, 2016 and U.S. Pat. No. 9,044,610, entitled “SYSTEM AND METHODS FOR PROVIDING A DISTRIBUTED VIRTUAL STIMULATION CATHODE FOR USE WITH AN IMPLANTABLE NEUROSTIMULATION SYSTEM” issued Jun. 02, 2015, which are hereby incorporated by reference. The IMD may monitor transthoracic impedance, such as implemented by the CorVue algorithm offered by St. Jude Medical. Additionally or alternatively, the IMD may include one or more structural and/or functional aspects of the device(s) described in U.S. Pat. No. 9,216,285, entitled “LEADLESS IMPLANTABLE MEDICAL DEVICE HAVING REMOVABLE AND FIXED COMPONENTS” issued Dec. 22, 2015 and U.S. Pat. No. 8,831,747, entitled “LEADLESS NEUROSTIMULATION DEVICE AND METHOD INCLUDING THE SAME” issued Sep. 09, 2014, which are hereby incorporated by reference. Additionally or alternatively, the IMD may include one or more structural and/or functional aspects of the device(s) described in U.S. Pat. No. 8,391,980, entitled “METHOD AND SYSTEM FOR IDENTIFYING A POTENTIAL LEAD FAILURE IN AN IMPLANTABLE MEDICAL DEVICE” issued Mar. 05, 2013 and U.S. Pat. No. 9,232,485, entitled “SYSTEM AND METHOD FOR SELECTIVELY COMMUNICATING WITH AN IMPLANTABLE MEDICAL DEVICE” issued Jan. 05, 2016, which are hereby incorporated by reference. Additionally or alternatively, the IMD may be a subcutaneous IMD that includes one or more structural and/or functional aspects of the device(s) described in U.S. Application Serial No. 15/973,195, entitled “SUBCUTANEOUS IMPLANTATION MEDICAL DEVICE WITH MULTIPLE PARASTERNAL-ANTERIOR ELECTRODES” filed May 07, 2018; U.S. Application Serial No. 15/973,219, entitled “IMPLANTABLE MEDICAL SYSTEMS AND METHODS INCLUDING PULSE GENERATORS AND LEADS” filed May 07, 2018; U.S. Application Serial No. 15/973, 249, entitled “SINGLE SITE IMPLANTATION METHODS FOR MEDICAL DEVICES HAVING MULTIPLE LEADS”, filed May 07, 2018, which are hereby incorporated by reference in their entireties. Further, one or more combinations of IMDs may be utilized from the above incorporated patents and applications in accordance with embodiments herein. Embodiments may be implemented in connection with one or more subcutaneous implantable medical devices (S-IMDs). For example, the S-IMD may include one or more structural and/or functional aspects of the device(s) described in U.S. Application Serial No. 15/973,219, entitled “IMPLANTABLE MEDICAL SYSTEMS AND METHODS INCLUDING PULSE GENERATORS AND LEADS”, filed May 07, 2018; U.S. Application Serial No. 15/973,195, entitled “SUBCUTANEOUS IMPLANTATION MEDICAL DEVICE WITH MULTIPLE PARASTERNAL-ANTERIOR ELECTRODES”, filed May 07, 2018; which are hereby incorporated by reference in their entireties. The IMD may represent a passive device that utilizes an external power source, and entirely mechanical plan will device, and/or an active device that includes an internal power source. The IMD may deliver some type of therapy/treatment, provide mechanical circulatory support, and/or merely monitor one or more physiologic characteristics of interest (e.g., PAP, CA signals, impedance, heart sounds).

Additionally or alternatively, embodiments herein may be implemented in connection with an integrated healthcare patient management system or network, such as described in U.S. Published Application 20210020294, entitled “METHODS, DEVICE AND SYSTEMS FOR HOLISTIC INTEGRATED HEALTHCARE PATIENT MANAGEMENT” filed Jul. 16, 2020, which is incorporated by reference herein in its entirety.

Additionally or alternatively, embodiments herein may be implemented in combination with the processes and structures described in U.S. Pat. 11,221,373, titled “Method and Device for Detecting Early Battery Depletion Condition”, issuing Jan. 11, 2022, U.S. Pat. 8,406,873, titled “Methods and Systems for implementing a high voltage switching circuit in an IMD”, issuing Mar. 26, 2013, U.S. Pat. 11,211,805, titled “Methods, systems and devices that estimate longevity of an implantable medical device”, issuing Dec. 28, 2021, U.S. Pat. 6,096,062, titled “Method and apparatus for maintaining a high voltage capacitor in an implantable cardiac device”, issuing Aug. 01, 2000, US 6,169,923 Pat., titled “Implantable cardioverter-defibrillator with automatic arrhythmia detection criteria adjustment”, issuing Jan. 02, 2001, U.S. Pat. 7,050,850, titled “Methods and apparatus for reforming high-voltage electrolytic capacitors”, issuing May 23, 2006, U.S. Pat. 7,917,217, titled “Wet tantalum reformation method and apparatus”, issuing Mar. 29, 2011, U.S. Pat. 8,036,740, titled “Wet-tantalum reformation method and apparatus”, issuing Oct. 11, 2011, and U.S. Pat. 8,346,355, titled “Capacitor reformation method and apparatus”, issuing Jan. 01, 2013, which are incorporated by reference herein in their entirety.

The term “health care system” shall mean a system that includes equipment for measuring health parameters, and communication pathways from the equipment to secondary devices. The secondary devices may be at the same location as the equipment, or remote from the equipment at a different location. The communication pathways may be wired, wireless, over the air, cellular, in the cloud, etc. In one example, the healthcare system provided may be one of the systems described in U.S. Provisional Pat. App. No. 62/875,870 entitled METHODS DEVICE AND SYSTEMS FOR HOLISTIC INTEGRATED HEALTHCARE PATIENT MANAGEMENT, to Rupinder, filed Jul. 18, 2019, the entire contents of which are incorporated in full by reference herein. Other Pats. that describe example monitoring systems include U.S. Pat. No. 6,572,557; entitled SYSTEM AND METHOD FOR MONITORING PROGRESSION OF CARDIAC DISEASE STATE USING PHYSIOLOGIC SENSORS, filed Dec. 21, 2000, to Tchou et al.; U.S. Pat. No. 6,480,733 entitled METHOD FORMONITORING HEART FAILURE filed Dec. 17, 1999, to Turcott; U.S. Pat. No. 7,272,443 entitled SYSTEM AND METHOD FOR PREDICTING A HEART CONDITION BASED ON IMPEDANCE VALUES USING AN IMPLANTABLE MEDICAL DEVICE, filed Dec. 14, 2004, to Min et al; U.S. Pat. No. 7,308,309 entitled DIAGNOSING CARDIAC HEALTH UTILIZING PARAMETER TREND ANALYSIS, filed Jan. 11, 2005, to Koh; and U.S. Pat. No. 6,645,153 entitled SYSTEM AND METHOD FOR EVALUATING RISK OF MORTALITY DUE TO CONGESTIVE HEART FAILURE USING PHYSIOLOGIC SENSORS, filed Feb. 7, 2002, to Kroll et. al., the entire contents of which are incorporated in full by reference herein.

All references, including publications, patent applications and patents, cited herein are hereby incorporated by reference to the same extent as if each reference were individually and specifically indicated to be incorporated by reference and were set forth in its entirety herein.

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

Some or all of the Figures herein illustrate various methods and processes implemented in accordance with embodiments herein. The operations herein may be implemented by hardware, firmware, circuitry and/or one or more processors housed partially an/or entirely within an IMD, a local external device, remote server or more generally within a healthcare system. Optionally, the operations herein may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage media having computer (device) readable program code embodied thereon.

Any combination of at least one non-signal computer (device) readable medium may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It should be recognized that, to the extent embodiments herein are described to apply certain mathematical combinations of select variables, the same variables may be combined in other mathematical combinations that are also indicative of the same result. For example, when a single data point is utilized for a particular variable, additionally or alternatively, a mean, average, sum, or other mathematical combination of multiple data points may be utilized for the same variable.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc., are used merely as labels, and are not intended to impose numerical requirements on their objects or order of execution on their acts.

What is claimed is:

1. A system, comprising:
a charge storage device within an implantable medical device (IMD);
an energy supply configured to charge the charge storage device during a charging operation;
a monitoring circuit coupled to the charge storage device, the monitoring circuit configured to collect voltage level measurements across the charge storage device at multiple points in time during the charging operation;
a processor that, when executing program instructions, is configured to:
collect the voltage level measurements across the charge storage device, wherein the charge storage device exhibits a charge profile in which a voltage level across the charge storage device changes over the charging operation;
analyze the voltage level measurements to identify a failure signature in the charge profile; and
in response to identifying the failure signature, generate an output indicative of a failure state for the charge storage device based on the analysis.

2. The system of claim 1, wherein the failure signature represents a partial discharge in the charge profile, wherein the partial discharge is expressed by a decrease in the voltage level measurements, followed by a recovery in the voltage level measurements, while actively charging the charge storage device.

3. The system of claim 1, wherein, when the charge storage device is in a non-failure state, the charge profile exhibiting a voltage level that monotonically increases throughout a period of time in which the charge storage device is actively being charged, and, when the charge storage device is in a failure state, the charge profile exhibiting one or more voltage drops during the period of time in which the charge storage device is actively being charged.

4. The system of claim 1, wherein, to analyze the voltage level measurements, the processor is further configured to calculate a rate of change per unit of time in the charge profile and to determine when the rate of change has a negative slope.

5. The system of claim 1, wherein, to analyze the voltage level measurements, the processor is further configured to calculate voltage changes between successive first and second voltage level measurements and to determine when the second voltage level measurement is below the first voltage level measurement.

6. The system of claim 1, wherein the charge profile further includes an active segment and an inactive segment, the active segment corresponding to a period of time during the charging operation in which the charge storage device is actively being charged, the inactive segment corresponding to a period of time during the charging operation in which the charge storage device is not being charged.

7. The system of claim 1, wherein the IMD further houses the energy supply and the monitoring circuit.

8. The system of claim 7, further comprising an external device configured to wirelessly communicate with the IMD, at least one of the IMD or the external device including a memory and the processor.

9. The system of claim 7, wherein the output includes a communication from the IMD, the communication indicating that one or more capacitors of the charge storage device is experiencing the failure state.

10. The system of claim 7, wherein the charge storage device includes one or more capacitors in the IMD, the failure state for the one or more capacitors includes at least one of i) a loss of a dielectric layer of a capacitor, ii) corrosion detected, iii) oxide formation, iv) at least one of iron, chlorine, bromine, iodine, or copper contamination, or v) short circuit between adjacent dielectric layers.

11. A computer implemented method, comprising:
under control of one or more processors, configured with specific executable instructions,
utilizing an energy supply to charge a charge storage device, within an implantable medical device (IMD), during a charging operation;
directing a monitoring circuit to collect voltage level measurements across the charge storage device at multiple points in time during the charging operation, wherein the charge storage device exhibits a charge profile in which a voltage level across the charge storage device changes over the charging operation;
analyzing the voltage level measurements to identify a failure signature in the charge profile; and
in response to identifying the failure signature, generating an output indicative of a failure state for the charge storage device based on the analysis.

12. The method of claim 11, wherein the failure signature represents one of an increase in time or a decrease in time for the charge profile to reach a predetermined voltage level compared to a baseline charge time to reach the predetermined voltage level.

13. The method of claim 12, further comprising:
adjusting the time for the charge profile to reach the predetermined voltage level based on at least one of voltage and equivalent series resistance associated with the energy supply; and
comparing the adjusted time to the baseline charge time.

14. The method of claim 11, wherein the failure signature represents a partial discharge in the charge profile, wherein the partial discharge is expressed by a decrease in the voltage level measurements, followed by a recovery in the voltage level measurements, while actively charging the charge storage device.

15. The method of claim 11, wherein, when the charge storage device is in a non-failure state, the charge profile exhibiting a voltage level that monotonically increases throughout a period of time in which the charge storage device is actively being charged, and, when the charge storage device is in a failure state, the charge profile exhibiting one or more voltage drops during the period of time in which the charge storage device is actively being charged.

16. The method of claim 11, wherein the analyzing the voltage level measurements further comprises calculating a rate of change per unit of time in the charge profile and determining when the rate of change has a negative slope.

17. The method of claim 11, wherein the analyzing the voltage level measurements further comprises calculating voltage changes between successive first and second voltage level measurements and determining when the second voltage level measurement is below the first voltage level measurement.

18. The method of claim 11, wherein the charge profile further includes an active segment and an inactive segment, the active segment corresponding to a period of time during the charging operation in which the charge storage device is actively being charged, the inactive segment corresponding to a period of time during the charging operation in which the charge storage device is not being charged, the method further comprising:
directing the monitoring circuit to collect second voltage level measurements across the charge storage device at multiple points during the inactive segment; and
analyzing the second voltage level measurements to identify a second failure signature in the charge profile.

19. The method of claim 18, wherein the second failure signature represents an increased rate of change compared to a baseline rate of change.

20. The method of claim 18, further comprising:
directing the monitoring circuit to collect third voltage level measurements across the charge storage device at multiple points during a second inactive segment that is subsequent to the inactive segment; and
averaging the second and third voltage level measurements across the inactive segment and the second inactive segment.
